SEQUENCE LISTING (8 sequences)

SEQ ID NO: 1 - DNA, 15 bp, Artificial Sequence
  Description of Artificial Sequence Synthetic primer
cggacagcgccagcg

SEQ ID NO: 2 - DNA, 22 bp, Artificial Sequence
  Description of Artificial Sequence Synthetic primer
gcattattacagtccaccagcg

SEQ ID NO: 3 - DNA, 20 bp, Artificial Sequence
  Description of Artificial Sequence Synthetic primer
aggtggatgcaagatcaagg

SEQ ID NO: 4 - DNA, 20 bp, Artificial Sequence
  Description of Artificial Sequence Synthetic primer
tagccagttgtggctttgtg

SEQ ID NO: 5 - DNA, 20 bp, Artificial Sequence
  Description of Artificial Sequence Synthetic primer
cggtgtgattgaaggtggat

SEQ ID NO: 6 - DNA, 20 bp, Artificial Sequence
  Description of Artificial Sequence Synthetic primer
acaggaaacgcaccatatcc

SEQ ID NO: 7 - DNA, 18 bp, Artificial Sequence
  Description of Artificial Sequence Synthetic primer
cccatgactcccatgacc

SEQ ID NO: 8 - DNA, 23 bp, Artificial Sequence
  Description of Artificial Sequence Synthetic primer
tttacaaccaagattcactgtgg

(19) United States
(12) Patent Application Publication
(10) Pub. No.: US 2021/0308134 A1
(43) Pub. Date: Oct. 7, 2021

(54) TREATMENT OF EGFR-MUTANT CANCER

(71) Applicants: BLUEPRINT MEDICINES CORPORATION, Cambridge, MA (US); The General Hospital Corporation, Boston, MA (US)

(72) Inventors: Aaron Hata, Boston, MA (US); Lecia Sequist, Boston, MA (US); Beni B. Wolf, Cambridge, MA (US)

(21) Appl. No.: 17/267,149

(22) PCT Filed: Aug. 9, 2019
(86) PCT No.: PCT/US2019/045919

§ 371 Date: Feb. 9, 2021

Related U.S. Application Data
(60) Provisional application No. 62/735,730, filed on Sep. 24, 2018.
(60) Provisional application No. 62/717,480, filed on Aug. 10, 2018.

(51) Int. Cl. A61K 31/506; A61K 9/00; A61P 35/00; A61K 31/47

(52) U.S. Cl. CPC A61K 31/506; A61K 31/47; A61P 35/00; A61K 9/0056

(57) ABSTRACT

Disclosed herein are methods for treating an EGFR-mutant cancer in a patient in need thereof by administering to the patient a therapeutically effective amount of at least one RET inhibitor (e.g., Compound 1 and/or pharmaceutically acceptable salts thereof) and a therapeutically effective amount of at least one EGFR inhibitor (e.g., osimertinib and/or pharmaceutically acceptable salts thereof), as well as combination therapies including at least one RET inhibitor and at least one EGFR inhibitor.

This application is a U.S. National Stage Application of International Patent Application No. PCT/US2019/045919, filed on Aug. 9, 2019, which claims priority to and the benefit of U.S. Patent Application No. 62/717,480, filed on Aug. 10, 2018; and U.S. Patent Application No. 62/735,730, filed on Sep. 24, 2018; the contents of each of which are incorporated by reference herein in their entirety.

This invention was made with Government support under Grant Numbers CA137008 and CA197389 awarded by the National Institutes of Health. The Government has certain rights in this invention.

### SEQUENCE LISTING

The instant application contains a Sequence Listing which has been submitted electronically in ASCII format and is hereby incorporated by reference in its entirety. Said ASCII copy, created on Feb. 12, 2021, is named BPM-6034WOUS_ST25.txt and is 2,151 bytes in size.

This disclosure relates to methods for treating an EGFR-mutant cancer in a patient in need thereof by administering to the patient a therapeutically effective amount of at least one RET inhibitor, e.g., at least one selective RET inhibitor, and a therapeutically effective amount of at least one EGFR inhibitor to the patient. For example, the disclosure relates to methods for treating an EGFR-mutant cancer in a patient who has been previously treated with at least one EGFR inhibitor, and, in some cases, has acquired resistance to the at least one EGFR inhibitor. This disclosure also relates to combination therapies comprising at least one RET inhibitor, e.g., at least one selective RET inhibitor, and at least one EGFR inhibitor. In some embodiments, the at least one RET inhibitor is a selective RET inhibitor chosen from Compound 1 and pharmaceutically acceptable salts thereof. In some embodiments, the at least one EGFR inhibitor is chosen from osimertinib and pharmaceutically acceptable salts thereof.

The receptor tyrosine kinase (RTK) rearranged during transfection (RET), along with glial cell line-derived neurotrophic factors (GDNF) and GDNF family receptors-α (GFRα), is required for the development, maturation, and maintenance of several neural, neuroendocrine, and genitourinary tissue types. However, increasing evidence implicates aberrant activation of RET as a critical driver of tumor growth and proliferation across a broad number of solid tumors (Mulligan L M., Nat. Rev. Cancer. 14:173-186 (2014)).

Oncogenic RET rearrangements have been identified in 1-2% of NSCLC (Lipson, D. et al., Nat. Med. 18:382-384 (2012); Takeuchi, K. et al., Nat. Med. 18:378-381 (2012); Stransky, N. et al., Nat. Commun. 5:4846 (2014)). Oncogenic RET rearrangements generate a constitutively active kinase that promotes tumorigenesis. As with anaplastic lymphoma kinase (ALK) and c-ros oncogene (ROS) 1-rearranged NSCLC, RET-rearranged NSCLC typically has adenocarcinoma histology (though occasionally squamous) and occurs in young, non-smoking patients. (1S,4R)—N—((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexanecarboxamide (Compound 1) described herein is a potent and selective inhibitor of RET kinase and oncogenic RET mutants. In cellular systems, Compound 1 inhibits the kinase activity of RET oncogenic mutants with low nanomolar potency. In vivo dose-dependent antitumor efficacy with Compound 1 has been demonstrated in several RET-driven models. Notably, in first-in-human testing, Compound 1 induced durable clinical responses in NSCLC patients with RET-alterations without notable off-target toxicity (Subbiah, V. et al. Cancer Disc (Apr. 15, 2018 early online release)). Compound 1 is currently being investigated for use in the treatment of patients with RET-driven malignancies such as thyroid cancer, non-small cell lung cancer (NSCLC), and other advanced solid tumors.

RET fusions may also be implicated in some cases of EGFR-mutant cancer (see Schrock, A. B. et al., J. Thorac. Oncol. doi: 10.1016/j.jtho.2018.05.027 (published online Jun. 5, 2018)). While certain EGFR inhibitors have been approved in the treatment of cancer, e.g., non-small cell lung cancer (osimertinib), a subset of patients progressing on EGFR inhibitor therapy have acquired gene fusions that cause acquired drug resistance. RET fusions associated with EGFR TKI resistance (see Oxnard, G. R. et al., JAMA Oncology doi:10.1001/jamaoncol.2018.2969 (published online Aug. 2, 2018) and Karen L. Reckamp et al., Analysis of Cell-Free DNA from 32,991 Advanced Cancers Reveals Novel Co-Occurring Activating RET Alterations and Oncogenic Signaling Pathway Aberrations at AACR Annual Meeting 2018 (Apr. 15, 2018)), such as CCDC6-RET, most frequently occur in the setting of “loss” of a prior documented T790M gatekeeper mutation of EGFR.

EGFR TKI resistance facilitated by RET fusions resembles the bypass track resistance facilitated by MET amplification in EGFR-mutant patients. In cases with MET amplification, both preclinical and clinical evidence have shown strong responses with by inhibiting both EGFR and MET (Engleman, J. A. et al, Science 316:1039-43 (2007); Gainor, J. F. et al, J. Thorac. Oncol. 11(7):e83-e85 (2016); Ahn, M. et al, J. Thorac. Oncol. 12(11S2):pS1768 (2017)).

However, for many patients with EGFR TKI resistance, treatment options are very limited, and the cancer progresses, unchecked, in most instances. Thus, despite the efficacy of EGFR inhibitors, including EGFR TKIs, as a monotherapy or a dual inhibitor therapy (with a MET inhibitor) in certain cancers, as well as the potential of RET inhibitors in certain cancers, there remains a need for even more effective treatment protocols in cancer.

### SUMMARY

The following disclosure describes methods for treating an EGFR-mutant cancer in a patient in need thereof by administering to the patient a therapeutically effective amount of at least one RET inhibitor and a therapeutically effective amount of at least one EGFR inhibitor. For example, in some embodiments, the patient has been previously treated with at least one EGFR inhibitor. In some embodiments, the patient has acquired resistance to at least one EGFR inhibitor.

Illustratively, in some embodiments, the at least one RET inhibitor is a selective RET inhibitor, e.g., Compound 1 or a pharmaceutically acceptable salt thereof. In some embodiments, Compound 1 is orally administered to the patient once daily. In some embodiments, the therapeutically effective amount of the at least one RET inhibitor administered to the patient once daily is 200 mg to 400 mg of Compound 1 or the weight equivalent of a pharmaceutically acceptable salt thereof. In some embodiments, the therapeutically effective amount of the at least one RET inhibitor administered to the patient once daily is 200 mg to 300 mg of Compound 1 or the weight equivalent of a pharmaceutically acceptable salt thereof.

In some embodiments, the at least one RET inhibitor is chosen from alectinib, apatinib, BOS172738 (DS-5010), cabozantinib (XL184), dovitinib (TKI258), GSK3179106, GSK3352589, lenvatinib, LOXO-292, SL-1001, TPX-0046, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.

In some embodiments, the at least one RET inhibitor is chosen from alectinib, apatinib, BOS172738 (DS-5010), cabozantinib (XL184), dovitinib (TKI258), GSK3179106, GSK3352589, lenvatinib, LOXO-292, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.

In some embodiments, the at least one EGFR inhibitor is chosen from osimertinib and pharmaceutically acceptable salts thereof. In some embodiments, osimertinib and/or at least one pharmaceutically acceptable salt thereof is orally administered to the patient once daily. In some embodiments, the therapeutically effective amount of the at least one EGFR inhibitor administered to the patient once daily is 80 mg of osimertinib or the weight equivalent of a pharmaceutically acceptable salt thereof.

In some embodiments, the EGFR-mutant cancer is characterized by at least one EGFR mutation chosen from L858R, Δex19, T790M, C797S, and L792H. Additionally, in some embodiments, the EGFR-mutant cancer is characterized by at least one EGFR mutation chosen from T790M, C797S, and L792H. In some embodiments, the EGFR-mutant cancer is characterized by at least two EGFR mutations. In some embodiments, the EGFR-mutant cancer is characterized by three EGFR mutations. In some embodiments, the EGFR-mutant cancer is further characterized by at least one RET-alteration, e.g., a CCDC6-RET fusion. In some embodiments, the EGFR-mutant cancer is lung cancer, e.g., small cell lung cancer (SCLC) or non-small cell lung cancer (NSCLC).

The following disclosure also describes combination therapies comprising at least one RET inhibitor, e.g., at least one selective RET inhibitor such as Compound 1 and/or a pharmaceutically acceptable salt thereof, and at least one EGFR inhibitor, e.g., osimertinib and/or a pharmaceutically acceptable salt of any of the foregoing.

Treating a patient, e.g., a human, suffering from an EGFR-mutant cancer with at least one RET inhibitor in combination with at least one EGFR inhibitor may improve therapeutic outcomes in patients with acquired drug resistance to EGFR TKIs.

Example embodiments of the disclosure further include:

1. A method for treating an EGFR-mutant cancer in a patient in need thereof comprising administering to the patient a therapeutically effective amount of at least one RET inhibitor and a therapeutically effective amount of at least one EGFR inhibitor.
2. The method of embodiment 1, wherein the at least one RET inhibitor is chosen from Compound 1 and pharmaceutically acceptable salts thereof.
3. The method of embodiment 1, wherein the at least one RET inhibitor is chosen from alectinib, apatinib, BOS172738 (DS-5010), cabozantinib (XL184), dovitinib (TKI258), GSK3179106, GSK3352589, lenvatinib, LOXO-292, SL-1001, TPX-0046, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.
4. The method of embodiment 1, wherein the at least one RET inhibitor is a selective RET inhibitor.
5. The method of any one of embodiments 1 to 4, wherein the at least one EGFR inhibitor is a selective EGFR inhibitor.
6. The method of any one of embodiments 1 to 4, wherein the at least one EGFR inhibitor is a third generation EGFR inhibitor.
7. The method of any one of embodiments 1 to 4, wherein the at least one EGFR inhibitor is chosen from osimertinib and pharmaceutically acceptable salts thereof.
8. The method of any one of embodiments 1 to 7, wherein the EGFR-mutant cancer is characterized by at least one EGFR mutation chosen from T790M, C797S, and L792H.
9. The method of any one of embodiments 1 to 8, wherein the EGFR-mutant cancer is further characterized by at least one RET-fusion.
10. The method of embodiment 9, wherein the EGFR-mutant cancer is further characterized by CCDC6-RET fusion.
11. The method of any one of embodiments 1 to 10, wherein the EGFR-mutant cancer is lung cancer.
12. The method of embodiment 11, wherein the lung cancer is chosen from small cell lung cancer and non-small cell lung cancer.
13. The method of any one of embodiments 1 to 12, wherein the patient is a human
14. The method of any one of embodiments 1 to 13, wherein the patient has been previously treated with at least one EGFR inhibitor.
15. The method of any one of embodiments 1 to 14, wherein the patient has acquired resistance to at least one EGFR inhibitor.
16. The method of any one of embodiments 1, 2, and 4-15, wherein:

the at least one RET inhibitor is chosen from Compound 1 and pharmaceutically acceptable salts thereof;

the at least one RET inhibitor is orally administered to the patient once daily; and

the therapeutically effective amount of the at least one RET inhibitor is 200 mg to 400 mg of Compound 1 or the weight equivalent of a pharmaceutically acceptable salt thereof.

17. The method of embodiment 16, wherein the therapeutically effective amount of the at least one RET inhibitor is 200 mg to 300 mg of Compound 1 or the weight equivalent of a pharmaceutically acceptable salt thereof.
18. The method of any one of embodiments 7 to 17, wherein

the at least one EGFR inhibitor is chosen from osimertinib and pharmaceutically acceptable salts thereof;

the at least one EGFR inhibitor is orally administered to the patient once daily; and

the therapeutically effective amount of the at least one EGFR inhibitor is 80 mg of osimertinib or the weight equivalent of a pharmaceutically acceptable salt thereof.

19. A combination therapy comprising at least one RET inhibitor and at least one EGFR inhibitor.
20. The combination therapy of embodiment 19, wherein the at least one RET inhibitor is chosen from Compound 1 and pharmaceutically acceptable salts thereof.
21. The combination therapy of embodiment 19, wherein the at least one RET inhibitor is chosen from alectinib, apatinib, BOS172738 (DS-5010), cabozantinib (XL184), dovitinib (TKI258), GSK3179106, GSK3352589, lenvatinib, LOXO-292, SL-1001, TPX-0046, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.
22. The combination therapy of embodiment 19, wherein the at least one RET inhibitor is a selective RET inhibitor.
23. The combination therapy of any one of embodiments 19 to 22, wherein the at least one EGFR inhibitor is a selective EGFR inhibitor.
24. The combination therapy of any one of embodiments 19 to 22, wherein the at least one EGFR inhibitor is a third generation EGFR inhibitor.
25. The combination therapy of embodiment 20, wherein Compound 1 is present in an amount of 200 mg to 400 mg.
26. The combination therapy of embodiment 20, wherein Compound 1 is present in an amount of 200 mg to 300 mg.
27. The combination therapy of any one of embodiments 19 to 22, 25, or 26, wherein the at least one EGFR inhibitor is chosen from osimertinib and pharmaceutically acceptable salts thereof.
28. The combination therapy of embodiment 27, wherein osimertinib is present in an amount of 80 mg.
29. A method for treating a patient suffering from an EGFR-mutant cancer, the method comprising:

(a) obtaining a biological sample from the patient;

(b) detecting the presence or absence of at least one RET-fusion in the biological sample; and

(c) administering a combination therapy to the patient if at least one RET-fusion is detected, wherein the combination therapy comprises at least one EGFR inhibitor and at least one RET inhibitor.

30. The method of embodiment 29, wherein the at least one RET inhibitor is chosen from Compound 1 and pharmaceutically acceptable salts thereof.
31. The method of embodiment 29, wherein the at least one RET inhibitor is chosen from alectinib, apatinib, BOS172738 (DS-5010), cabozantinib (XL184), dovitinib (TKI258), GSK3179106, GSK3352589, lenvatinib, LOXO-292, SL-1001, TPX-0046, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.
32. The method of embodiment 29, wherein the at least one RET inhibitor is a selective RET inhibitor.
33. The method of any one of embodiments 29 to 32, wherein the at least one EGFR inhibitor is chosen from osimertinib and pharmaceutically acceptable salts thereof.
34. The method of any one of embodiments 29 to 32, wherein the at least one EGFR inhibitor is a selective EGFR inhibitor.
35. The method of any one of embodiments 29 to 32, wherein the at least one EGFR inhibitor is a third generation EGFR inhibitor.
36. The method of any one of embodiments 29 to 35, wherein the EGFR-mutant cancer is characterized by at least one EGFR mutation chosen from T790M, C797S, and L792H.
37. The method of any one of embodiment 29 to 36, wherein the at least one RET-fusion is a CCDC6-RET fusion.
38. The method of any one of embodiments 29 to 37, wherein the EGFR-mutant cancer is lung cancer.
39. The method of embodiment 38, wherein the lung cancer is chosen from small cell lung cancer and non-small cell lung cancer.
40. The method of any one of embodiments 29 to 39, wherein the patient is a human.
41. The method of any one of embodiments 29 to 40, wherein the patient has been previously treated with at least one EGFR inhibitor.
42. The method of any one of embodiments 29 to 41, wherein the patient has acquired resistance to at least one EGFR inhibitor.

The above embodiments are provided to introduce a selection of the concepts discussed herein. These embodiments are not intended to identify essential features of the disclosed subject matter or to limit the scope of the disclosed subject matter.

### BRIEF DESCRIPTION OF THE FIGURES

FIG. 1 is a scan which shows RECIST tumor shrinkage of 78% in a 60-year-old woman with EGFR dell9 and acquired CCDC6-RET fusion after two weeks of treatment with 200 mg once daily of Compound 1 and 80 mg once daily of osimertinib, followed by six weeks of treatment with 300 mg once daily of Compound 1 and 80 mg once daily of osimertinib. The serial coronal contrast-enhanced computed-tomography images of the thorax demonstrate a right lower lobe lung mass and pleural nodularity (arrows) seen at baseline (left) with partial response after eight weeks of treatment with Compound 1 and osimertinib (right).

FIG. 2 demonstrates that CCDC6-RET expressing cell line models were generated by lentiviral infection of PC9 (EGFR del19) and MGH134 (EGFR L858R/T790M) cells. CCDC6-RET fusion gene or internal control TBP transcripts from LC2/ad cells and PC9^{CCDC6-RET} or MGH134^{CCDC6-RET} cells were amplified by RT-PCR.

FIG. 3 shows cell proliferation data for PC9^{CCDC6-RET} and MGH134^{CCDC6-RET} cells in the presence or absence of osimertinib. PC9 and MGH134 cells expressing the CCDC6-RET gene fusion or empty vector (EV) were treated with 1 μM osimertinib or vehicle (VEH) and cell proliferation determined over the course of five days (ratio compared to the beginning of treatment). Data shown are the mean±s.e.m. of three independent biological replicates.

FIG. 4A is a western blot for PC9^{EV} and PC9^{CCDC6-RET} cells treated with 100 nM afatinib, 1 μM osimertinib, Compound 1, or combinations thereof for 6 hours and harvested for western blotting with the antibodies.

FIG. 4B is a western blot for MGH134^{EV} and MGH134^{CCDC6-RET} cells treated with 1 μM osimertinib, cabozantinib, and Compound 1 or combinations thereof for 6 hours and harvested for western blotting with the indicated antibodies.

FIG. 4C shows cell viability after 72 hours for PC9^{EV} and PC9^{CCDC6-RET} cells treated with Compound 1, or afatinib or osimertinib in the absence or presence of 1 μM Compound 1. The same Compound 1 data is replotted in both panels for comparison purposes. Data are shown as a percentage of vehicle treated control and are the mean±s.e.m of three independent biological replicates.

FIG. 5A is a western blot for PC^{9V} and PC9^{CCDC6-RET} cells treated with 100 nM afatinib, 1 μM osimertinib, cabozanitinib, or combinations thereof for 6 hours and harvested for western blot analysis.

FIG. 5B shows cell viability after 72 hours for PC9^{EV} and PC^{CCDC6-RET} cells treated with cabozantinib, or afatinib or osimertinib in the absence or presence of 1 μM cabozantinib (CAB). The same cabozantinib data is replotted in both panels for comparison purposes. Data are shown as a percentage of vehicle treated control and are the mean±s.e.m of three independent biological replicates.

FIG. 5C shows cell viability after 72 hours for MGH134^{EV} and MGH134^{CCDC6-RET} cells treated with a RET inhibitor, cabozantinib, or Compound 1, or osimertinib in the absence or presence of 1 μM RET inhibitor. Data are shown as a percentage of vehicle treated control and are the mean±s.e.m of three independent biological replicates.

### EMBODIMENTS OF THE DISCLOSURE

### Abbreviations and Definitions

The following abbreviations and terms have the indicated means throughout:

As used herein, a “combination therapy” refers to a therapy comprising more than one active agent. The two or more active agents can be administered in one dosage form or separate dosage forms. Additionally, the active agents comprising the combination therapy may be administered at the same time (in one or more dosage forms) or at separate times.

“Compound 1” is (1S,4R)—N—((S)-1-(6-(4-fluoro-1H-pyrazol-1-yl)pyridine-3-yl)ethyl)-1-methoxy-4-(4-methyl-6-((5-methyl-1H-pyrazol-3-yl)amino)pyrimidin-2-yl)cyclohexanecarboxamide:

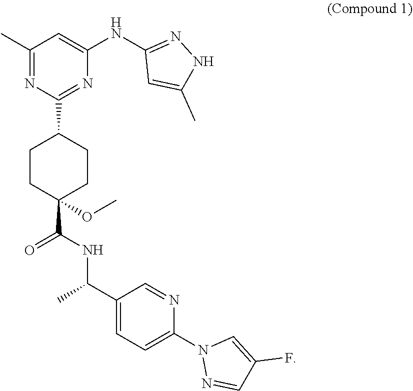 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

In March 2017, Compound 1 (also known as BLU-667 or pralsetinib) entered Phase I clinical trials in the United States for the treatment of patients with thyroid cancer, non-small cell lung cancer, and other advanced solid tumors (NCT03037385). WO 2017/079140, incorporated herein by reference, describes the synthesis of Compound 1 (Example Compound 130) and also discloses the therapeutic activity of this molecule to inhibit, regulate, and/or modulate RET kinase (Assays, Example 10 on pp. 72-74).

As used herein, an “EGFR inhibitor” is a compound which inhibits the activity of EGFR kinase. EGFR kinase is wild-type EGFR kinase and/or one or more EGFR-altered kinases (e.g., EGFR fusion, EGFR mutation, or EGFR copy number variation).

Examples of EGFR inhibitors include, but are not limited to, afatinib, ASP8273, avitinib, brigatinib, cetuximab, dacomitinib, EAI045, erlotinib, gefitinib, HS-10296, icotinib, lapatinib, necitumumab, nazartinib, neratinib, olmutinib, osimertinib, panitumumab, PF-06747775, rociletinib, and vandetanib.

As used herein, a “fusion” is a protein that results from a chromosomal translocation in which two genes are joined with an in-frame coding sequence and results in a chimeric protein. In some embodiments, a fusion is a chromosomal translocation where the kinase domain of one protein fuses to a dimerization domain of another gene.

As used herein, a “RET fusion” is a gene rearrangement. RET rearrangements create a fusion protein juxtaposing the RET kinase domain and a dimerization domain of another protein, creating a constitutively activated dimer which drives tumorigenesis.

As used herein, a “RET fusion protein” is the result of a gene rearrangement. RET rearrangements create a fusion protein juxtaposing the RET kinase domain and a dimerization domain of another protein, creating a constitutively activated dimer which drives tumorigenesis.

As used herein, a “RET inhibitor” is a compound which inhibits the activity of RET kinase. RET kinase is wild-type RET kinase and/or one or more RET-altered kinases (e.g., RET fusion, RET mutation, or RET copy number variation).

Examples of RET inhibitors include, but are not limited to, alectinib, cabozantinib (XL184), Compound 1, dovitinib (TKI258), BOS172738 (DS-5010), foretinib, lenvatinib, LOXO-292, ponatinib, RXDX-105, sitravatinib (MGCD516), sorafenib, sunitinib, TAS0286, TPX-0046, SL-1001, and vandetanib. Additional examples of RET inhibitors include, but are not limited to, apatinib, AUY-922, DCC-2157, GSK3179106, GSK3352589, motesanib, nintendanib, NVP-AST487, PZ-1, regorafenib (BAY 73-4506), RPI-1, TG101209, SPP86, vatalanib, and XL999.

Examples of RET inhibitors include, but are not limited to, alectinib, cabozantinib (XL184), Compound 1, dovitinib (TKI258), BOS172738 (DS-5010), foretinib, lenvatinib, LOXO-292, ponatinib, RXDX-105, sitravatinib (MGCD516), sorafenib, sunitinib, TAS0286, and vandetanib. Additional examples of RET inhibitors include, but are not limited to, apatinib, AUY-922, DCC-2157, GSK3179106, GSK3352589, motesanib, nintendanib, NVP-AST487, PZ-1, regorafenib (BAY 73-4506), RPI-1, TG101209, SPP86, vatalanib, and XL999.

As used herein, a “selective RET inhibitor” refers to a compound or a pharmaceutically acceptable salt thereof that selectively inhibits a RET kinase over another kinase and exhibits at least a 2-fold selectivity for a RET kinase over another kinase. For example, a selective RET inhibitor exhibits at least a 10-fold selectivity; at least a 15-fold selectivity; at least a 20-fold selectivity; at least a 30-fold selectivity; at least a 40-fold selectivity; at least a 50-fold selectivity; at least a 60-fold selectivity; at least a 70-fold selectivity; at least a 80-fold selectivity; at least a 90-fold selectivity; at least 100-fold, at least 125-fold, at least 150-fold, at least 175-fold, or at least 200-fold selectivity for a RET kinase over another kinase. In some embodiments, a selective RET inhibitor exhibits at least 20-fold selectivity over another kinase, e.g., JAK1. In some embodiments, a selective RET inhibitor exhibits at least 50-fold selectivity over another kinase, e.g. VEGFR-2 or TRKC. In some embodiments, a selective RET inhibitor exhibits at least 100-fold selectivity over another kinase, e.g., FLT3, JAK2, TRKA, or PDGFRβ. In some embodiments, a selective RET inhibitor exhibits at least 1000-fold selectivity over another kinase, e.g., LIMK1, FGFR1, c-SRC, ML2/MAP3K10, PEAK1, FGFR3, MLK3/MAP3K11, ROS/ROS1, c-KIT, YES/YES1, FLT4/VEGFR3, JAK3, or TYK2. In some embodiments, selectivity for a RET kinase over another kinase is measured in a cellular assay. In other embodiments, selectivity for a RET kinase over another kinase is measured in a biochemical assay.

Non-limiting examples of selective RET inhibitors include Compound I, SL-1001, and LOXO-292. Examples of selective RET inhibitors include Compound 1 and LOXO-292.

As used herein, the term “subject” or “patient” refers to organisms to be treated by the methods of the present disclosure. Such organisms include, but are not limited to, mammals (e.g., murines, simians, equines, bovines, porcines, canines, felines, and the like), and in some embodiments, humans.

Many cancers have been linked to EGFR mutations. Such cancers are referred to herein as “EGFR-mutant cancers.” EGFR-mutant cancers include lung cancers (e g, small cell lung cancer, non-small cell lung cancer, and squamous-cell carcinoma of the lung), anal cancers, colon cancers, thyroid cancers (e.g., papillary thyroid cancer), glioblastoma, epithelial cancers (e.g., epithelial tumors of the head and neck). In some embodiments, the EGFR-mutant cancer is characterized by at least one EGFR mutation chosen from T790M, C797S, and L792H. In some embodiments, the EGFR-mutant cancer is characterized by a T790M mutation. In some embodiments, the EGFR-mutant cancer is characterized by a C797S mutation. In some embodiments, the EGFR-mutant cancer is characterized by a L792H mutation. In some embodiments, the EGFR-mutant cancer is characterized by a L858R or Δex19 mutation. In some embodiments, the EGFR-mutant cancer is characterized by a L858R or Δex19 mutation and a T790M mutation. In some embodiments, the EGFR-mutant cancer is characterized by a L858R or Δex19 mutation and a C796S mutation. In some embodiments, the EGFR-mutant cancer is characterized by a L858R or Δex19 mutation, a T790M mutation, and a C796S mutation.

In some embodiments, the EGFR-mutant cancer is further characterized by at least one RET fusion (e.g., at least one RET fusion listed in Table 1). In some embodiments, the EGFR-mutant cancer is further characterized by CCDC6-RET fusion. In some embodiments, the EGFR-mutant cancer is further characterized by a KIF5B-RET fusion. In some embodiments, the EGFR-mutant cancer if further characterized by a NCOA4-RET fusion.

TABLE 1
RET Fusions.

| RET fusion partner | Example cancers in which the fusion is found |
|---|---|
| BCR | Chronic Myelomonocytic Leukemia (CMML) |
| CLIP 1 | Adenocarcinoma |
| KIF5B | NSCLC, Ovarian Cancer, Spitzoid Neoplasm; Lung |
| | Adenocarcinoma, Adenosquamous Carcinomas |
| CCDC6 | NSCLC, Colon Cancer, Papillary Thyroid Cancer; Adenocarcinoma; |
| | Lung Adenocarcinoma; Metastatic Colorectal Cancer; |
| | Adenosquamous Carcinoma, Metastatic papillary thyroid cancer |
| PTClex9 | Metastatic papillary thyroid cancer |
| NCOA4 | Papillary Thyroid Cancer, NSCLC, Colon Cancer, |
| | Salivary Gland Cancer, Metastatic Colorectal Cancer; Lung |
| | Adenocarcinoma, Adenosquamous Carcinomas; Diffuse Sclerosing |
| | Variant of Papillary Thyroid Cancer |
| TRIM33 | NSCLC, Papillary Thyroid Cancer |
| ERC1 | Papillary Thyroid Cancer, Breast Cancer |
| FGPRIOP | CMML, Primary Myelofibrosis with secondary Acute Myeloid |
| | Leukemia |
| MBD1 | Papillary Thyroid Cancer |
| RAB61P2 | Papillary Thyroid Cancer |
| PRKAR1A | Papillary Thyroid Cancer |
| TRIM24 | Papillary Thyroid Cancer |
| KTN1 | Papillary Thyroid Cancer |
| GOLGA5 | Papillary Thyroid Cancer, Spitzoid Neoplasms |
| HOOK3 | Papillary Thyroid Cancer |
| KIAA1468 | Papillary Thyroid Cancer, Lung Adenocarcinoma |
| TRIM27 | Papillary Thyroid Cancer |
| AKAP13 | Papillary Thyroid Cancer |
| FKBP15 | Papillary Thyroid Cancer |
| SPECC1L | Papillary Thyroid Cancer, Thyroid Gland Carcinoma |
| TBL1XR1 | Papillary Thyroid Cancer, Thyroid Gland Carcinoma |
| CEP55 | Diffuse Gastric Cancer |
| CUX1 | Lung Adenocarcinoma |
| ACBD5 | Papillary Thyroid Carcinoma |
| MYH13 | Medullary Thyroid Carcinoma |
| PIBF1 | Bronchiolus Lung Cell Carcinoma |
| KIAA1217 | Papillary Thyroid Cancer, Lung Adenocarcinoma, NSCLC |
| MPRIP | NSCLC |

Some of the RET fusions in Table 1 are discussed in: Grubbs et al., J Clin Endocrinol Metab, 100:788-93 (2015); Halkova et al., Human Pathology 46:1962-69 (2015); U.S. Pat. Nos. 9,297,011; 9,216,172; Le Rolle et al., Oncotarget 6(30):28929-37 (2015); Antonescu et al., Am J Surg Pathol 39(7):957-67 (2015); U.S. Patent Application Publication No. 2015/0177246; U.S. Patent Application Publication No. 2015/0057335; Japanese Patent Application Publication No. 2015/109806A; Chinese Patent Application Publication No. 105255927A; Fang, et al., Journal of Thoracic Oncology 11.2 (2016): S21-S22; European Patent Application Publication No. EP3037547A1; Lee et al., Oncotarget DOI: 10.18632/oncotarget.9137, e-published ahead of printing, 2016; Saito et al., Cancer Science 107:713-20 (2016); Pirker et al., Transl Lung Cancer Res, 4(6):797-800 (2015); and Joung et al., Histopathology 69(1):45-53 (2016).

A person of ordinary skill in the art may determine if a subject possesses a RET-fusion e.g., using a method selected from hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect a fusion, primary tumor samples may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumor samples.

Next, genes in which 5′ exons are expressed at significantly different levels than 3′ exons are identified. A sliding frame is used to identify the breakpoint within an individual sample. Specifically, at each iteration, an incremental breakpoint divides the gene into 5′ and 3′ regions, and a t-statistic is used to measure the difference in expression (if any) between the two regions. The breakpoint with the maximal t-statistic is chosen as the likely fusion breakpoint. As used herein, “breakpoint” is the boundary at which two different genes are fused. It is sometimes referred to as a “fusion point.” The location where the difference in exon expression is maximal between 5′ and 3′ is the inferred breakpoint of the fusion. Thousands of tumor samples can be rapidly profiled in this manner, generating a list of fusion candidates (ranked by t-statistic). High-ranking candidates can then be validated, and fusion partners identified by examining the raw RNA-seq data sets, and identifying chimeric pairs and/or split reads which support the fusion. Candidate fusions can then be experimentally confirmed as described below.

Alternatively, fusions may be identified by circulating tumor DNA (ctDNA) analysis of plasma (i.e., a liquid biopsy).

In addition, the methods described in Wang L et al., Genes Chromosomes Cancer 51(2):127-39 (2012). doi: 10.1002/gcc.20937, Epub 2011 Oct. 27; and Suehara Y et al., Clin Cancer Res. 18(24):6599-608 (2012). doi: 10.1158/1078-0432.CCR-12-0838, Epub 2012 Oct. 10 can also be used to detect a fusion.

In some embodiments of the disclosure, the EGFR-mutant cancer is a lung cancer. In some embodiments, the EGFR-mutant cancer is small cell lung cancer. In some embodiments, the EGFR-mutant cancer is non-small cell lung cancer. In some embodiments, the EGFR-mutant cancer is squamous-cell carcinoma of the lung.

In some embodiments, the EGFR-mutant cancer is anal cancer.

In some embodiments, the EGFR-mutant cancer is colon cancer.

In some embodiments, the EGFR-mutant cancer is thyroid cancer. In some embodiments, the EGFR-mutant cancer is papillary thyroid cancer.

In some embodiments, the EGFR-mutant cancer is glioblastoma.

In some embodiments, the EGFR-mutant cancer is epithelial cancer. In some embodiments, the EGFR-mutant cancer is an epithelial tumor of the head or neck.

In some embodiments, the patient suffering from an EGFR-mutant cancer has previously been treated with at least one EGFR inhibitor (e.g., osimertinib and/or pharmaceutically acceptable salts thereof). In some embodiments, the patient suffering from an EGFR-mutant cancer has acquired resistance to at least one EGFR inhibitor (e.g., osimertinib and/or pharmaceutically acceptable salts thereof).

As used herein, the phrase “therapeutically effective amount” refers to the amount of an active agent (e.g., Compound 1 or a pharmaceutically acceptable salt thereof) sufficient to effect beneficial or desired results. A therapeutically effective amount can be administered in one or more administrations, applications, or dosages and is not intended to be limited to a specific formulation or administration route.

As used herein, the phrase “weight equivalent of a pharmaceutically acceptable salt thereof” in reference to a specific compound includes the weight of both the compound and the associated salt. For example, TAGRISSO® tablets contain 47.7 or 95.4 mg of osimertinib mesylate, which is the weight equivalent of 40 or 80 mg of osimertinib, respectively.

As used herein, the phrase “pharmaceutically acceptable salt thereof,” if used in relation to an active agent distributed as a salt form, refers to any pharmaceutically acceptable salt form of the active agent. For example, pharmaceutically acceptable salts of osimertinib mesylate include osimertinib besylate, osimertinib hydrochloride, etc.

As used herein, the term “treating” includes any effect, e.g., lessening, reducing, modulating, ameliorating, or eliminating, that results in the improvement of the condition, disease, disorder, and the like, or ameliorating a symptom thereof.

RET inhibitors that may be utilized in some embodiments include those that are well-known in the art, e.g., alectinib, apatinib, AUY-922, cabozantinib (XL184), Compound 1, DCC-2157, dovitinib (TKI258), BOS172738 (DS-5010), foretinib, GSK3179106, GSK3352589, lenvatinib, LOXO-292, TPX-0046, SL-1001, motesanib, nintendanib, NVP-AST487, ponatinib, PZ-1, regorafenib (BAY 73-4506), RPI-1, RXDX-105, TG101209, sitravatinib (MGCD516), sorafenib, sunitinib, RPI-1, TAS0286, TG101209, SPP86, vatalanib, vandetanib, XL999, as well as compounds disclosed in PCT publications WO 2005/062795, WO 2007/087245, WO 2009/003136, WO 2009/100536, WO 2010/006432, WO 2014/039971, WO 2014/050781, WO 2014/141187, WO 2015/006875, WO 2015/079251, WO 2016/037578, WO 2016/038552, WO 2016/075224, WO 2016/127074, WO 2017/011776, WO 2017/079140, WO 2017/145050, WO 2017/161269, WO 2017/178844, WO 2017/178845, WO 2018/017983, WO 2018/022761, WO2018/064852, WO2018/060714, WO 2018/071454, WO 2018/071447, WO2018/102455, WO 2018/136661, WO 2018/136663, WO2018/189553, WO2018/136661, WO2019/001556, WO2019/008172, WO2019/126121, WO2019/143977, WO2019/143991 and WO2019/143994.

In some embodiments, the RET inhibitor is a multikinase inhibitor that was originally designed to target other kinases, e.g., vascular endothelial growth factor receptor 2 (VEGFR-2), tyrosine-protein kinase MET, and/or EGFR, that inhibits the other kinases more potently than RET, e.g., cabozantinib, vandetanib, sunitinib, levatinib, regorafenib, and RXDX-105. In some embodiments, multikinase inhibitors with RET activity are poor inhibitors of RET with gatekeeper mutations at the V804 residue, e.g., V804L and V804M.

In some embodiments, the at least one RET inhibitor is a selective RET inhibitor. In some embodiments, the selective inhibitor was designed for highly potent and selective targeting of wild-type (WT) RET and oncogenic mutant forms of RET, e.g., prevalent RET alterations, including RET fusions (e.g., KIF5B-RET, CCDC6-RET), and RET activating mutations (e.g., C634W, M918T, V804L/M), while maintaining selectivity against other human kinases. In some embodiments, a selective RET inhibitor has activity against multiple oncogenic mutant forms of RET, regardless of tumor type. In some embodiments, the equipotent activity of a selective RET inhibitor across multiple oncogenic mutant forms of RET differentiates the selective RET inhibitor from a multikinase inhibitor with RET inhibitory activity.

In some embodiments, the at least one RET inhibitor is a selective RET inhibitor. Compound 1 is a RET-selective inhibitor that only inhibits wild-type RET and one or more mutant forms of RET and has little inhibitory activity against other kinases. LOXO-292 (selpercatnib) is also a selective RET inhibitor. Selective RET inhibitors that may be utilized in some embodiments include those that are well-known in the art, e.g., compounds disclosed in WO 2016/127074, WO 2017/011776, WO 2017/079140, WO 2017/161269, WO 2018/017983, WO 2018/022761, WO 2018/071454, WO 2018/071447, WO 2018/136661, WO 2018/136663, WO2018/237134, WO2019/001556, WO2019/143994, WO2019/143991, and WO2019/143977.

For example, in some embodiments, the at least one RET inhibitor is a selective RET inhibitor chosen from:

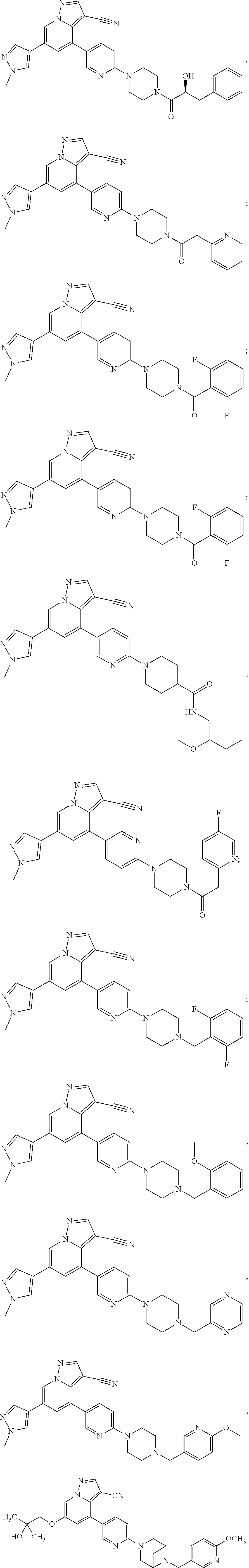

and pharmaceutically acceptable salts of any of the foregoing.

In some embodiments, the at least one RET inhibitor is a selective RET inhibitor chosen from: 4-(6-(4-benzylpiperazin-1-yl)pyridin-3-yl)-6-(2-morpholinoethoxy)pyrazolo[1,5-a]pyridine-3-carbonitrile; 6-(2-hydroxyethoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile; (R)-6-(2-hydroxypropoxy)-4-(6-(4-((6-methoxypyridin-3-yl)methyl)piperazin-1-yl)pyridin-3-yl)pyrazolo[1,5-a]pyridine-3-carbonitrile; 6-(2-hydroxy-2-methylpropoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile; 6-(2-methoxyethoxy)-4-6-(4-((6-methoxypyridin-3-yl)methyl)piperazin-1-yl)pyridin-3-yl)pyrazolo[1,5-a]pyridine-3-carbonitrile; 6-(2-hydroxy-2-methylpropoxy)-4-(6-(6-(6-methoxynicotinoyl)-3,6-diazabicyclo[3.1.1]heptan-3-yl)pyridin-3-yl)pyrazolo[1,5-a]pyridine-3-carbonitrile; 6-(2-(dimethylamino)ethoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile; 4-(6-(6-((6-methoxypyridin-3-yl)methyl)-3,6-diazabicyclo[3.1.1]heptan-3-yl)pyridin-3-yl)-6-(2-morpholinoethoxy)pyrazolo[1,5-a]pyridine-3-carbonitrile; 4-(6-(6-((6-methoxypyridin-3-yl)methyl)-3,6-diazabicyclo[3.1.1]heptan-3-yl)pyridin-3-yl)-6-((1-methyl-1H-imidazol-4-yl)methoxy)pyrazolo[1,5-a]pyridine-3-carbonitrile; 6-ethoxy-4-(5-(6-((5-fluoro-6-methoxypyridin-3-yl)methyl)-3,6-diazabicyclo[3.1.1]heptan-3-yl)pyrazin-2-yl)pyrazolo[1,5-a]pyridine-3-carbonitrile; and pharmaceutically acceptable salts of any of the foregoing.

In some embodiments, the at least one RET inhibitor is a selective RET inhibitor chosen from: N-(1-(5-(3-cyano-6-(2-hydroxy-2-methylpropoxy)pyrazolo[1,5-a]pyridin-4-yl)pyridin-2-yl)-4-methylpiperidin-4-yl)benzamide; 6-ethoxy-4-(6-(4-hydroxy-4-(pyridin-2-ylmethyl)piperidin-1-yl)pyridin-3-yl)pyrazolo[1,5-a]pyridine-3-carbonitrile; 6-(2-hydroxy-2-methylpropoxy)-4-(6-(3-(pyridin-2-yloxy)azetidin-1-yl)pyridin-3-yl)pyrazolo[1,5-a]pyridine-3-carbonitrile; 6-(2-hydroxy-2-methylpropoxy)-4-(6-(4-((6-methoxypyridazin-3-yl)oxy)piperidin-1-yl)pyridin-3-yl)pyrazolo[1, 5-a]pyridine-3-carbonitrile; (S)-6-(2-hydroxy-2-methylpropoxy)-4-(6-(3-(pyridin-2-yloxy)pyrrolidin-1-yl)pyridin-3-yl)pyrazolo[1, 5-a]pyridine-3-carbonitrile; N-(1-(5-(3-cyano-6-((3-fluoro-1-methylazetidin-3-yl)methoxy)pyrazolo [1,5-a]pyridin-4-yl)pyridin-2-yl)-4-methylpiperidin-4-yl)-5-fluoro-2-methylbenzamide; 3-chloro-N-(1-(5-(3-cyano-6-((3-fluoro-1-methylazetidin-3-yl)methoxy)pyrazolo[1,5-a]pyridin-4-yl)pyridin-2-yl)-4-methylpiperidin-4-yl)picolinamide; N-((3S,4S)-1-(5-(3-cyano-6-ethoxypyrazolo[1,5-a]pyridin-4-yl)pyridin-2-yl)-3-hydroxypiperidin-4-yl)-3-methylbutanamide; 6-(2-hydroxy-2-methyl propoxy)-4-(6-(4-hydroxy-4-(pyridin-2-ylmethyl)piperidin-1-yl)pyridin-3-yl)pyrazolo[1,5-a]pyridine-3-carbonitrile; 3-chloro-N-((3S,4S)-1-(5-(3-cyano-6-ethoxypyrazolo[1,5-a]pyridin-4-yl)pyrazin-2-yl)-3-hydroxypiperidin-4-yl)picolinamide; and pharmaceutically acceptable salts of any of the foregoing.

In some embodiments, the at least one RET inhibitor is administered once daily. In some embodiments, the at least one RET inhibitor is administered orally. In some embodiments, the at least one RET inhibitor is orally administered once daily.

In some embodiments, the at least one RET inhibitor is chosen from Compound 1 and pharmaceutically acceptable salts thereof. In some embodiments, the therapeutically effective amount of the at least one RET inhibitor orally administered once daily is 200 mg to 400 mg of Compound 1 or the weight equivalent of a pharmaceutically acceptable salt thereof. In some embodiments, the therapeutically effective amount of the at least one RET inhibitor orally administered once daily is 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, or 400 mg of Compound 1 or the weight equivalent of a pharmaceutically acceptable salt thereof.

In some embodiments, the at least one RET inhibitor is chosen from Compound 1 and pharmaceutically acceptable salts thereof. In some embodiments, the therapeutically effective amount of the at least one RET inhibitor orally administered once daily is 200 mg to 400 mg of Compound 1 or the weight equivalent of a pharmaceutically acceptable salt thereof. In some embodiments, the therapeutically effective amount of the at least one RET inhibitor orally administered once daily is 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg of Compound 1 or the weight equivalent of a pharmaceutically acceptable salt thereof.

In some embodiments, the at least one RET inhibitor is chosen from Compound 1 and pharmaceutically acceptable salts thereof. In some embodiments, the therapeutically effective amount of the at least one RET inhibitor orally administered once daily is 200 mg to 300 mg of Compound 1 or the weight equivalent of a pharmaceutically acceptable salt thereof. In some embodiments, the therapeutically effective amount of the at least one RET inhibitor orally administered once daily is 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg of Compound 1 or the weight equivalent of a pharmaceutically acceptable salt thereof.

In some embodiments, the therapeutically effective amount of the at least one RET inhibitor orally administered once daily is 200 mg of Compound 1 or the weight equivalent of a pharmaceutically acceptable salt thereof. In some embodiments, the therapeutically effective amount of the at least one RET inhibitor orally administered once daily is 205 mg of Compound 1 or the weight equivalent of a pharmaceutically acceptable salt thereof. In some embodiments, the therapeutically effective amount of the at least one RET inhibitor orally administered once daily is 210 mg of Compound 1 or the weight equivalent of a pharmaceutically acceptable salt thereof. In some embodiments, the therapeutically effective amount of the at least one RET inhibitor orally administered once daily is 215 mg of Compound 1 or the weight equivalent of a pharmaceutically acceptable salt thereof. In some embodiments, the therapeutically effective amount of the at least one RET inhibitor orally administered once daily is 220 mg of Compound 1 or the weight equivalent of a pharmaceutically acceptable salt thereof. In some embodiments, the therapeutically effective amount of the at least one RET inhibitor orally administered once daily is 225 mg of Compound 1 or the weight equivalent of a pharmaceutically acceptable salt thereof. In some embodiments, the therapeutically effective amount of the at least one RET inhibitor orally administered once daily is 230 mg of Compound 1 or the weight equivalent of a pharmaceutically acceptable salt thereof. In some embodiments, the therapeutically effective amount of the at least one RET inhibitor orally administered once daily is 235 mg of Compound 1 or the weight equivalent of a pharmaceutically acceptable salt thereof. In some embodiments, the therapeutically effective amount of the at least one RET inhibitor orally administered once daily is 240 mg of Compound 1 or the weight equivalent of a pharmaceutically acceptable salt thereof. In some embodiments, the therapeutically effective amount of the at least one RET inhibitor orally administered once daily is 245 mg of Compound 1 or the weight equivalent of a pharmaceutically acceptable salt thereof. In some embodiments, the therapeutically effective amount of the at least one RET inhibitor orally administered once daily is 250 mg of Compound 1 or the weight equivalent of a pharmaceutically acceptable salt thereof. In some embodiments, the therapeutically effective amount of the at least one RET inhibitor orally administered once daily is 255 mg of Compound 1 or the weight equivalent of a pharmaceutically acceptable salt thereof. In some embodiments, the therapeutically effective amount of the at least one RET inhibitor orally administered once daily is 260 mg of Compound 1 or the weight equivalent of a pharmaceutically acceptable salt thereof. In some embodiments, the therapeutically effective amount of the at least one RET inhibitor orally administered once daily is 265 mg of Compound 1 or the weight equivalent of a pharmaceutically acceptable salt thereof. In some embodiments, the therapeutically effective amount of the at least one RET inhibitor orally administered once daily is 270 mg of Compound 1 or the weight equivalent of a pharmaceutically acceptable salt thereof. In some embodiments, the therapeutically effective amount of the at least one RET inhibitor orally administered once daily is 275 mg of Compound 1 or the weight equivalent of a pharmaceutically acceptable salt thereof. In some embodiments, the therapeutically effective amount of the at least one RET inhibitor orally administered once daily is 280 mg of Compound 1 or the weight equivalent of a pharmaceutically acceptable salt thereof. In some embodiments, the therapeutically effective amount of the at least one RET inhibitor orally administered once daily is 285 mg of Compound 1 or the weight equivalent of a pharmaceutically acceptable salt thereof. In some embodiments, the therapeutically effective amount of the at least one RET inhibitor orally administered once daily is 290 mg of Compound 1 or the weight equivalent of a pharmaceutically acceptable salt thereof. In some embodiments, the therapeutically effective amount of the at least one RET inhibitor orally administered once daily is 295 mg of Compound 1 or the weight equivalent of a pharmaceutically acceptable salt thereof. In some embodiments, the therapeutically effective amount of the at least one RET inhibitor orally administered once daily is 300 mg of Compound 1 or the weight equivalent of a pharmaceutically acceptable salt thereof. In some embodiments, the therapeutically effective amount of the at least one RET inhibitor orally administered once daily is 305 mg of Compound 1 or the weight equivalent of a pharmaceutically acceptable salt thereof. In some embodiments, the therapeutically effective amount of the at least one RET inhibitor orally administered once daily is 310 mg of Compound 1 or the weight equivalent of a pharmaceutically acceptable salt thereof. In some embodiments, the therapeutically effective amount of the at least one RET inhibitor orally administered once daily is 315 mg of Compound 1 or the weight equivalent of a pharmaceutically acceptable salt thereof. In some embodiments, the therapeutically effective amount of the at least one RET inhibitor orally administered once daily is 320 mg of Compound 1 or the weight equivalent of a pharmaceutically acceptable salt thereof. In some embodiments, the therapeutically effective amount of the at least one RET inhibitor orally administered once daily is 325 mg of Compound 1 or the weight equivalent of a pharmaceutically acceptable salt thereof. In some embodiments, the therapeutically effective amount of the at least one RET inhibitor orally administered once daily is 330 mg of Compound 1 or the weight equivalent of a pharmaceutically acceptable salt thereof. In some embodiments, the therapeutically effective amount of the at least one RET inhibitor orally administered once daily is 335 mg of Compound 1 or the weight equivalent of a pharmaceutically acceptable salt thereof. In some embodiments, the therapeutically effective amount of the at least one RET inhibitor orally administered once daily is 340 mg of Compound 1 or the weight equivalent of a pharmaceutically acceptable salt thereof. In some embodiments, the therapeutically effective amount of the at least one RET inhibitor orally administered once daily is 345 mg of Compound 1 or the weight equivalent of a pharmaceutically acceptable salt thereof. In some embodiments, the therapeutically effective amount of the at least one RET inhibitor orally administered once daily is 350 mg of Compound 1 or the weight equivalent of a pharmaceutically acceptable salt thereof. In some embodiments, the therapeutically effective amount of the at least one RET inhibitor orally administered once daily is 355 mg of Compound 1 or the weight equivalent of a pharmaceutically acceptable salt thereof. In some embodiments, the therapeutically effective amount of the at least one RET inhibitor orally administered once daily is 360 mg of Compound 1 or the weight equivalent of a pharmaceutically acceptable salt thereof. In some embodiments, the therapeutically effective amount of the at least one RET inhibitor orally administered once daily is 365 mg of Compound 1 or the weight equivalent of a pharmaceutically acceptable salt thereof. In some embodiments, the therapeutically effective amount of the at least one RET inhibitor orally administered once daily is 370 mg of Compound 1 or the weight equivalent of a pharmaceutically acceptable salt thereof. In some embodiments, the therapeutically effective amount of the at least one RET inhibitor orally administered once daily is 375 mg of Compound 1 or the weight equivalent of a pharmaceutically acceptable salt thereof. In some embodiments, the therapeutically effective amount of the at least one RET inhibitor orally administered once daily is 380 mg of Compound 1 or the weight equivalent of a pharmaceutically acceptable salt thereof. In some embodiments, the therapeutically effective amount of the at least one RET inhibitor orally administered once daily is 385 mg of Compound 1 or the weight equivalent of a pharmaceutically acceptable salt thereof. In some embodiments, the therapeutically effective amount of the at least one RET inhibitor orally administered once daily is 390 mg of Compound 1 or the weight equivalent of a pharmaceutically acceptable salt thereof. In some embodiments, the therapeutically effective amount of the at least one RET inhibitor orally administered once daily is 395 mg of Compound 1 or the weight equivalent of a pharmaceutically acceptable salt thereof. In some embodiments, the therapeutically effective amount of the at least one RET inhibitor orally administered once daily is 400 mg of Compound 1 or the weight equivalent of a pharmaceutically acceptable salt thereof.

In addition, in some embodiments, the at least one EGFR inhibitor is chosen from afatinib, ASP8273, avitinib, brigatinib, cetuximab, dacomitinib, EAI045, erlotinib, gefitinib, HS-10296, icotinib, lapatinib, necitumumab, nazartinib, neratinib, olmutinib, osimertinib, panitumumab, PF-06747775, EGF816, YH5448, avitinib, rociletinib, vandetanib, and pharmaceutically acceptable salts of any of the foregoing.

In some embodiments, the at least one EGFR inhibitor is a selective inhibitor. In some embodiments, a selective EGFR inhibitor was designed for highly potent and selective targeting of oncogenic mutant forms of EGFR, e.g., exon 19 deletion, L858R, T790M. In some embodiments, a selective EGFR inhibitor has activity against multiple oncogenic mutant forms of EGFR, regardless of tumor type. In some embodiments, the equipotent activity of a selective EGFR inhibitor across multiple oncogenic mutant forms of EGFR differentiates a selective EGFR inhibitor from a multikinase inhibitor with EGFR inhibitory activity.

In some embodiments, a selective EGFR inhibitor is a third generation EGFR inhibitor. In some embodiments, a selective EGFR inhibitor has activity against oncogenic mutant forms of EGFR, including exon 19 deletion, L858R, and T790M. In some embodiments, a selective EGFR inhibitor includes osimertinib, rociletinib, olmutinib, EGF816, PF-06747775, YH5448, and avitinib. In some embodiments, the selective EGFR inhibitor does not have activity against WT EGFR. In some embodiments, a selective EGFR inhibitor is not a covalent inhibitor.

In some embodiments, the at least one EGFR inhibitor is administered once daily. In some embodiments, the at least one EGFR inhibitor is administered orally. In some embodiments, the at least one EGFR inhibitor is orally administered once daily.

In some embodiments, the at least one EGFR inhibitor is chosen from osimertinib and pharmaceutically acceptable salts thereof. In some embodiments, the therapeutically effective amount of the at least one EGFR inhibitor orally administered once daily is 80 mg of osimertinib or the weight equivalent of a pharmaceutically acceptable salt thereof.

While it is possible for an active agent (e.g., Compound 1 or osimertinib) to be administered alone, in some embodiments, the active agent can be administered as a pharmaceutical formulation, wherein the active agent is combined with one or more pharmaceutically acceptable excipients or carriers. For example, the active agent may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of pharmaceutically acceptable carriers include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Solid dosage forms (e.g., capsules, tablets, pills, dragees, powders, granules, and the like) can include one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

Liquid dosage forms can include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an active compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Dosage forms for the topical or transdermal administration of Compound 1 include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required.

When Compound 1 is administered as a pharmaceutical, to humans and animals, it can be given per se or as a pharmaceutical composition containing, for example, 0.1 to 99.5% (such as 0.5 to 90%) of active ingredient in combination with a pharmaceutically acceptable carrier.

The formulations can be administered topically, orally, transdermally, rectally, vaginally, parentally, intranasally, intrapulmonary, intraocularly, intravenously, intramuscularly, intraarterially, intrathecally, intracapsularly, intradermally, intraperitoneally, subcutaneously, subcuticularly, or by inhalation.

The present disclosure is further illustrated by the following examples, which should not be construed as further limiting. The contents of all references cited throughout this application are expressly incorporated herein by reference.

### EXAMPLES

### Reagents and Antibodies

Afatinib, osimertinib, and cabozantinib were purchased from Selleck Chemicals and resuspended in DMSO. Phospho-EGFR (Y1068), EGFR, pBRAF (Ser445), RET, pAKT (Ser473), AKT, pMEK1/2 (Ser217/221), MEK1/2, pERK1/2 (Thr202/204), ERK1/2, and Actin antibodies were purchased from Cell Signaling Technology. pRET (Y1062) antibody was obtained from Abcam.

### RT-PCR and Sequencing

Total RNA from cell lines was extracted using an RNeasy Mini Kit (Qiagen). RNA (1 μg) was reverse transcribed using SuperScript™ II Reverse Transcriptase (Invitrogen), according to the manufacturer's instructions. CCDC6-RET, PCBP2-BRAF, and TBP were PCR amplified using the following primers: CCDC6 exon 1 F-CGGACAGCGCCAGCG (SEQ ID NO: 1), RET exon 19 R-GCATTATTACAGTCCACCAGCG (SEQ ID NO: 2); (PCBP2-BRAF Primer 1) PCBP2 exon 6 F-AGGTGGATGCAAGATCAAGG (SEQ ID NO: 3), BRAF exon 13 R-TAGCCAGTTGTGGCTTTGTG (SEQ ID NO: 4); (PCBP2-BRAF Primer 2) PCBP2 exon 2 F-CGGTGTGATTGAAGGTGGAT (SEQ ID NO: 5), BRAF exon 18 R-ACAGGAAACGCACCATATCC (SEQ ID NO: 6); TBP F-CCCATGACTCCCATGACC (SEQ ID NO: 7), TBP R-TTTACAACCAAGATTCACTGTGG (SEQ ID NO: 8). The PCR products were confirmed by agarose gel electrophoresis. Following amplification, Sanger sequencing was performed.

### Cell Culture

The PC9 and MGH134 cell lines are known in the art (Hata, A. N. et al., Nat. Med. 22(3):262-69 (2016)). Cells were cultured in RPMI1640 (Life Technologies) supplemented with 10% FBS. MGH845-1 cells were additionally cultured in 150 nM osimertinib. All cells were routinely tested and verified to be free of mycoplasma contamination.

### Generation of CCDC6-RET Expressing Cell Lines

A CCDC6-RET fusion construct was synthesized by GenScript and ligated into the pLENTI6/V5-D-TOPO vector using the ViraPower Lentiviral Directional TOPO Expression Kit (Life Technologies). Lentivirus was generated by transfecting the pLENTI6 constructs and packaging plasmids into 293FT cells (Life Technologies). Virus production, collection, and infection were completed following the manufacturer's protocol. Transduced cells were selected in blasticidin (10-20 mg/mL) for one week.

### Cell Viability Assay

For drug dose-response assays, cells were seeded into 96-well plates 24 hours before addition of drug. Cell proliferation was determined by CellTiter-Glo assay (Promega) 72-120 hours after adding drug, using standard protocols. For time-course experiments, multiple plates were seeded and drugged in identical fashion, and at the indicated time points, the plates were frozen at −80° C.; all plates in an experiment were developed with CellTiter-Glo simultaneously. Luminescence was measured with SpectraMax i3x Multi-Mode Microplate Reader (Molecular Devices).

### Example 1: Osimertinib AR Biopsies

To better characterize acquired resistance (AR) to osimertinib, a single-center cohort of osimertinib AR biopsies was performed. Osimertinib AR biopsies were assayed via SNaPshot or Foundation One next-generation sequencing (NGS) and plasma via Guardant360 NGS under an IRB-approved protocol. Specifically, forty-one EGFR-mutant patients treated with osimertinib for T790M+ disease were queried by tissue NGS (n=22), plasma NGS (n=9), or both (n=10). In two out of thirty-two tissue samples, SCLC transformation was observed. In five of the tissue samples and five of the plasma samples (all cis with T790M), EGFR C797S was found. Additionally, MET amplification was observed in seven tissue and three plasma samples. BRAF rearrangement was identified in two of the thirty-two tissue samples, while CCDC6-RET rearrangement was found in one of the thirty-two tissue samples, as well as one of the nineteen plasma samples. The tissue and plasma samples exhibiting CCDC6-RET rearrangement came from separate donors, indicating that RET rearrangements are a low frequency but recurrent finding in EGFR-mutant patients with AR to osimertinib.

### Example 2: Patient Studies

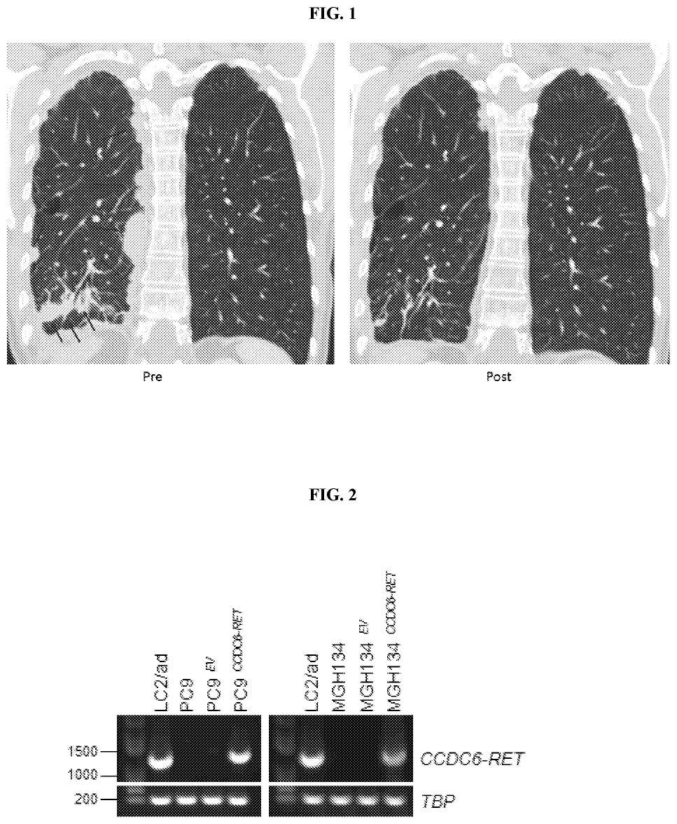

A 60-year-old woman with del19 EGFR-mutant advanced NSCLC received front-line afatinib (one year), acquired T790M, and was treated with osimertinib (18 months). She then underwent a lung biopsy revealing a CCDC6-RET fusion via SFA. Baseline tissue was insufficient for a solid fusion assay (SFA), but RET fluorescence in situ hybridization (FISH) was negative, suggesting the CCDC6-RET fusion was acquired. An individual patient investigational new drug (IND) protocol was written for the patient for treatment with osimertinib plus Compound 1. She began osimertinib 80 mg daily and Compound 1 200 mg daily, then increased Compound 1 to 300 mg after 2 weeks of treatment. Her dyspnea improved within days of therapy initiation. Scans after 8 weeks revealed a marked response with RECIST tumor shrinkage of 78% (FIG. 1). The combination was well-tolerated with only grade 1 toxicities, including fatigue, leukopenia, hypertension, dry mouth, and elevated transaminases. Treatment is ongoing as of Sep. 24, 2018.

A 44-year-old man with dell9 EGFR-mutant advanced NSCLC who received front-line cisplatin/pemetrexed, second-line afatinib (one year) underwent a bronchoscopic biopsy of a growing lung lesion showing a CCDC6-RET fusion by SFA. Baseline tissue was not available for RET testing. He was treated with erlotinib 150 mg daily combined with off-label cabozantinib 60 mg daily. Scans after one month showed stable disease (RECIST 1.1), but subsequent scans after 2.5 months showed disease progression and prompted treatment discontinuation. The patient had grade 1 diarrhea, rash, and AST elevation

### Example 3: CCDC6-RET Expression in EGFR-Mutant NSCLC Cell Lines Confers Resistance to EGFR Inhibitors

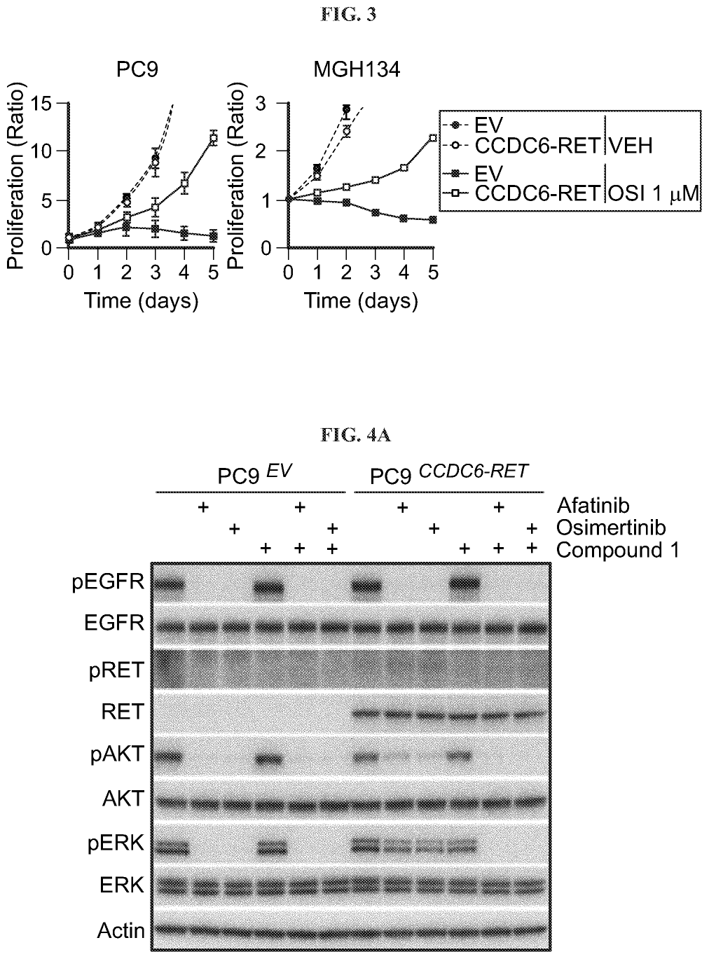

To determine whether CCDC6-RET expression is sufficient to cause acquired drug resistance, CCDC6-RET fusion expressing cell line models were generated by lentiviral infection of PC9 (EGFR del19) and MGH134 (EGFR L858R/T790M) cells (FIG. 2).

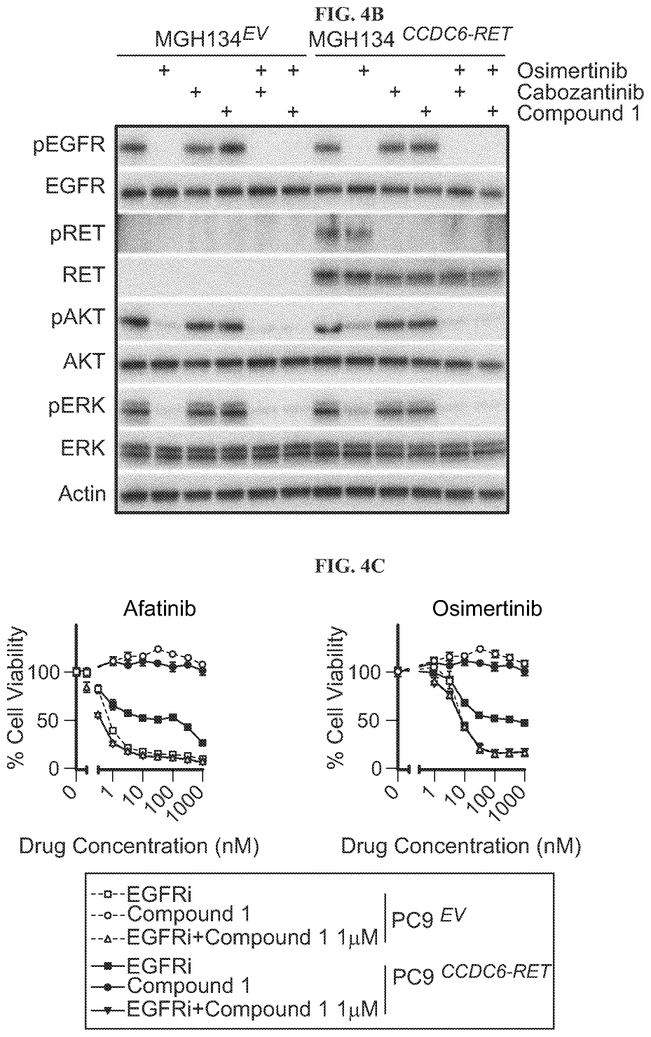

Cells expressing CCDC6-RET grew similarly to parental cells in the absence of EGFR inhibitor. When treated with osimertinib (OSI), PC9^{CCDC6-RET} and MGH134^{CCDC6-RET} cells continued to proliferate, in contrast to parental cells (EV), which experienced a net decrease in cell viability (FIG. 3). The proliferation rate of CCDC6-RET expressing cells decreased with osimertinib treatment, suggesting that RET activation does not fully compensate for EGFR signaling loss, although it is sufficient to drive acquired resistance.

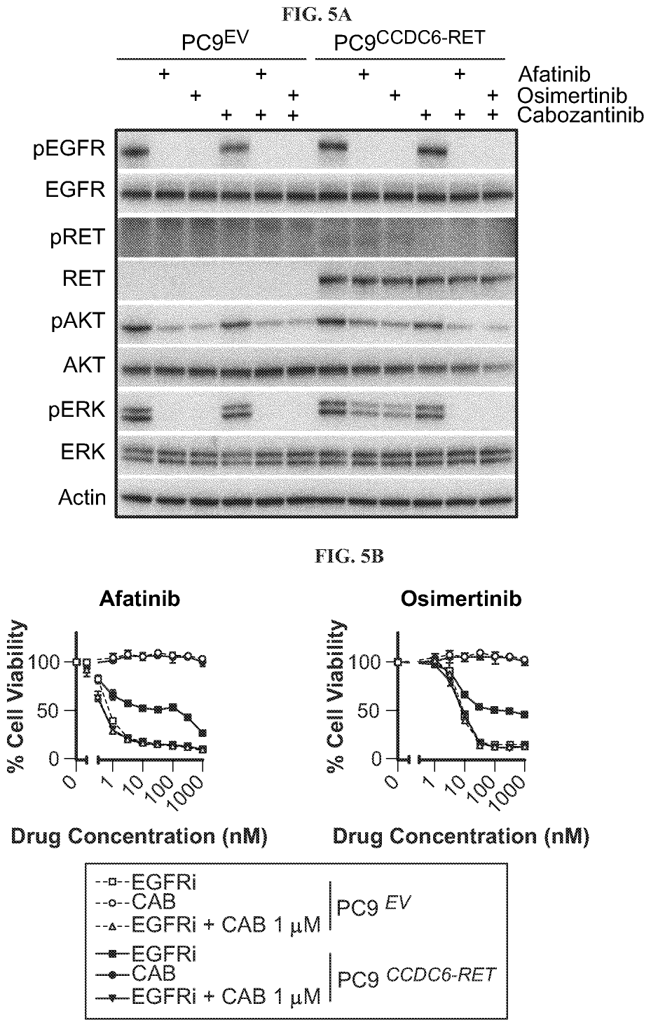

The consequences of CCDC6-RET expression on downstream signaling pathway activation in PC9 and MGH134 cells was also examined. Compared to parental cells, which did not express detectable RET protein, phosphorylated RET was detected in both PC9^{CCDC6-RET} and MGH134^{CCDC6-RET} cells (FIGS. 4A-4B). CCDC6-RET expression alone did not lead to increased activation of downstream MAPK (phospho-ERK1/2) or PI3K (phospho-AKT) signaling at baseline; however, RET, ERK1/2, and AKT phosphorylation was retained in the presence of afatinib or osimertinib in both PC9^{CCDC6-RET} and MGH134^{CCDC6-RET} cells (FIGS. 4A-4B). Thus, expression of the CCDC6-RET fusion can confer resistance to EGFR-inhibitors in EGFR-mutant NSCLCs.

### Example 4: Acquired Resistance Resulting from CCDC6-RET Expression can be Overcome by EGFR Plus RET

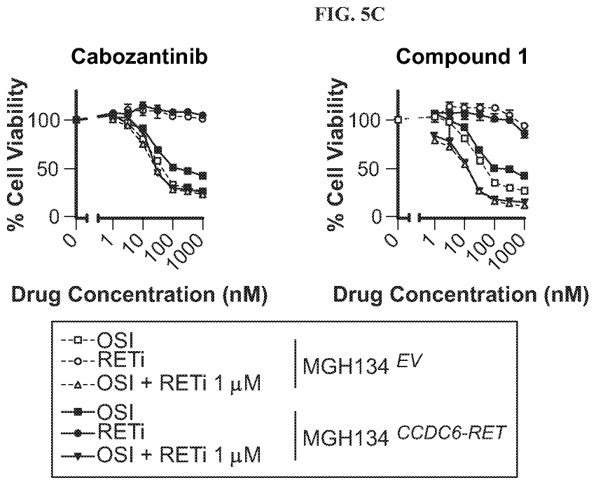

PC9CCDC6-RET cells, generated as above, were treated with Compound 1 in the absence or presence of EGFR inhibitors. Treatment with Compound 1 alone suppressed RET phosphorylation, but did not decrease downstream ERK or AKT phosphorylation (FIG. 4A). Combined treatment with Compound 1 and either osimertinib or afatinib completely suppressed both phospho-ERK and phospho-AKT and decreased cell viability to a similar level as parental cells treated with EGFR TKI (FIG. 4C). Similar results were observed in MGH134^{CCDC6-RET} cells (FIGS. 4B, 5C). Additionally, PC9^{CCDC6-RET} and MGH134^{CCDC6-RET} cells were sensitive to EGFR TKI+cabozantinib, a multi-kinase inhibitor with RET activity (FIGS. 4B, 5A-5C). Taken together, these data demonstrate that acquired resistance resulting from the CCDC6-RET fusions can be overcome by dual EGFR plus RET blockade.

### Example 5: Study of Compound 1 and Osimertinib for Metastatic Non-Small Cell Lung Cancer with RET Fusion-Mediated Resistance to EGFR Inhibition (Combination Study for Metastatic NSCLC with RET-Mediated Resistance to EGFR Inhibition)

This study is an open-label, Phase 1/2 study designed to evaluate the safety, tolerability, antitumor activity, PK, and pharmacodynamics of the potent and selective RET inhibitor, Compound 1, in combination with the third-generation EGFR inhibitor, osimertinib, in patients with NSCLC that have developed a RET fusion in association with resistance to osimertinib.

A dose-escalation study is conducted to assess the safety and tolerability of the combination of Compound 1 and osimertinib, and to identify the maximum tolerated dose (MTD) and/or recommended Phase 2 dose (RP2D). The overall safety profile of combination treatment with Compound 1 and osimertinib, is assessed by the type, frequency, severity, timing, and relationship to study drug of any adverse events, serious adverse events, changes in vital signs, ECGs, and safety laboratory tests.

The study also estimates the overall response rate (ORR) for Compound 1 and osimertinib combination therapy in patients with metastatic, RET fusion-positive, non-small cell lung cancer that has progressed during or after osimertinib. The ORR is defined as the proportion of patients who achieve a confirmed complete response (CR) or partial response (PR) according to RECIST 1.1.

The study also assesses additional measures of anticancer activity, including duration of response (DOR), disease control rate (DCR), clinical benefit rate (CBR), progression-free survival (PFS), and overall survival (OS). In addition, the study correlates Compound 1 PK parameters with safety endpoints and antitumor activity; further characterizes the safety and tolerability of the combination of Compound 1 and osimertinib; and assesses changes in measures of quality of life (QoL) and symptom severity.

For the additional measures, DOR is defined as the number of months from the time criteria are first met for either CR or PR, until the first date that progressive disease (PD) is objectively documented for patients with confirmed CR or PR; DCR is defined as the proportion of patients who experience a best response of stable disease (SD), partial response (PR), or complete response (CR) according to RECIST 1.1; partial response (PR), or complete response (CR) according to RECIST 1.1; and PFS is defined as the number of months from first dose of study treatment to the earlier of PD or death due to any cause.

PK parameters of Compound 1 include: population-derived estimates including maximum plasma drug concentration (Cₘₐₓ), area under the plasma concentration versus time curve from time 0 to 24 hours postdose (AUC₀₋₂₄), plasma drug concentration at 24 hours post-dose (C₂₄) at steady-state; and type, frequency, severity, timing, and relationship to study drug of any AEs, serious adverse events (SAEs), changes in vital signs, ECGs, and safety laboratory tests.

The study includes a standard 3+3 dose-escalation portion to identify the MTD and/or RP2D of Compound 1 when given in combination with osimertinib, followed by an expansion phase to assess ORR and other measures of clinical activity. All patients enrolled in the Phase 1 study portion must begin osimertinib treatment at the approved starting dose of 80 mg/day. In the Phase 2 study portion, patients who experienced toxicity with prior osimertinib treatment may initiate osimertinib at a lower starting dose if necessary. The Compound 1 dose levels are evaluated in dose escalation of 200 mg, 300 mg, and 400 mg. Patients in the expansion phase receive the RP2D as determined from dose-escalation. The expansion portion follows a 2-stage design, in which initially 10 patients are treated. If ≥2/10 patients from this first stage experience an objective tumor response, the second stage enrolls an additional 23 patients, for a total of 33 patients treated in the expansion phase.

For study eligibility, RET fusion status is determined by local or central assessment using a tumor or blood sample taken at the time of (or following) progression of disease on an EGFR inhibitor.

Study treatments, Compound 1 and osimertinib, are given by daily oral administration, as 28-day cycles. Dose modifications are according to specific criteria based on observed toxicities.

Patients may continue to receive study treatment until precluded by toxicity, noncompliance, withdrawal of consent, death, or closure of the study. Patients who experience RECIST 1.1-defined progression of disease but continue to experience clinical benefit in the opinion of the treating investigator may continue study therapy with approval. If Compound 1 is permanently discontinued, the patient is considered to have completed the study treatment period; and other anticancer therapy (including, if appropriate, osimertinib) is received as subsequent therapy during survival follow-up. Patients who require permanent discontinuation of osimertinib may continue Compound 1 monotherapy after approval.

All study visits are intended to be conducted on an outpatient basis, but may be conducted on an inpatient basis, as needed. Disease assessments are performed every 8 weeks for the first two years, then every 12 weeks thereafter. Following discontinuation of study treatment, patients without documented progressive disease continue to undergo disease assessments until documentation of progressive disease, initiation of another antineoplastic therapy, death, or closure of the study. Tumor response is assessed in accordance with RECIST 1.1. Patients also are contacted 30 days after discontinuation of study treatment for an assessment of safety, and continue survival follow-up until death or closure of the study.

The patient population includes participants that: are ≥18 years of age at the time of signing the informed consent; have pathologically confirmed, definitively diagnosed, metastatic EGFR-mutant NSCLC; have at least one target lesion evaluable by RECIST 1.1; for Phase 1 only: have radiologically documented disease progression during or after previous treatment with any 2nd or 3rd generation EGFR inhibitor TKI; for Phase 2 only: have radiologically documented disease progression during or after previous treatment with osimertinib; have oncogenic RET fusion, as detected by local or central testing of tumor tissue or circulating tumor nucleic acid in blood, using a sample as described above (for patients with RET status determined locally for eligibility, the patient must also consent to submission of blood and tissue samples for retrospective confirmation of RET status by central testing); are willing to provide archived tumor tissue (if a sample obtained following progression of disease during or after previous treatment with osimertinib is available) or, if appropriate archived tumor tissue is not available, is willing to undergo a pretreatment biopsy and the investigator considers the pretreatment biopsy safe and medically feasible (if performed after the baseline radiographic imaging, pretreatment biopsies are taken from a nontarget lesion); and an Eastern Cooperative Oncology Group (ECOG) performance status (PS) of 0-1.

The patient population excludes participants that: have any additional known primary driver alteration (other than the original EGFR mutation and RET fusion), including but not limited to targetable mutations of ALK, ROS1, MET, and BRAF; have any past medical history of interstitial lung disease (ILD) or interstitial pneumonitis, radiation pneumonitis that required steroid treatment, or any evidence of clinically active ILD within 28 days prior to enrollment; have central nervous system (CNS) metastases or a primary CNS tumor that is associated with progressive neurological symptoms or requires increasing doses of corticosteroids to control the CNS disease (if a patient requires corticosteroids for management of CNS disease, the dose must have been stable for the 2 weeks preceding C1D1); have had any anti-PD-1/PDL-1/CTLA therapy within 6 months, and any other anticancer therapy (including both systemic therapy and radiotherapy) within 14 days or 5 half-lives prior to the first dose of study drug, whichever is shorter (excluding prior osimertinib, which may be continued uninterrupted without a wash-out); have had more than 30 Gy of radiotherapy to the lung in the 6 months prior to enrollment; have QTcF>480 msec, a history of prolonged QT syndrome or Torsades de pointes, or a familial history of prolonged QT syndrome; or have any of the following within 14 days prior to the first dose of study drug:
a. Platelet count <75×10⁹/L;
b. Absolute neutrophil count (ANC)<1.0×10⁹/L;
c. Hemoglobin <9.0 g/dL (red blood cell transfusion and erythropoietin may be used to reach at least 9.0 g/dL, but must have been administered at least 2 weeks prior to the first dose of study drug;
d. Aspartate aminotransferase (AST) or alanine aminotransferase (ALT)>3×the upper limit of normal (ULN) if no hepatic metastases are present; >5×ULN if hepatic metastases are present;
f. Estimated (Cockroft-Gault formula) or measured creatinine clearance <40 mL/min; or
g. Total serum phosphorous >5.5 mg/dL.

A sample size of up to 18 patients is included in the Phase 1 dose-escalation phase. The total number of participants enrolled in the dose-escalation is dependent upon the observed safety profile, which determines the number of participants per dose cohort as well as the number of cohorts required to confirm the recommended Phase 2 dose (RP2D).

For the Phase 2 expansion phase, using a Simon's two-stage design (Simon, 1989), the sample size at stage 1 is 10 patients (30% of the total sample size) by assuming the null hypothesis response rate 5% and the alternative response rate 25% with one sided alpha 0.025 and power 90%. The cumulative sample size from stage 1 and stage 2 is 33. At stage 1, the trial is stopped due to failure to reject the null hypothesis if the response rate is no more than 1/10 (10%). Otherwise, the trial continues to stage 2. The null hypothesis is rejected if there are at least 5 responders among all 33 patients.

With 33 patients, there is >95% probability of observing at least one AE that occurs at a frequency of 10%; there is >99% probability of observing at least one AE that occurs at a frequency of 20%.

For the analysis population, the Response Evaluable Population (REP) includes all patients who have measurable disease at baseline, receive at least one dose of each study treatment (Compound 1 and osimertinib), and have an evaluable post-baseline tumor response assessment. The REP is utilized for the primary analyses of ORR, DCR and CBR. The Safety Population, including all patients who receive at least one dose of Compound 1, is utilized for the remaining efficacy endpoints and safety.

The number and percentage of patients with objective response are presented for the REP along with the 2-sided 95% confidence interval using Exact Clopper Pearson methodology. DCR and CBR are estimated along with 2-sided 95% confidence interval based on the same approach. For patients who achieve an objective response, the DOR is calculated from the time that the CR/PR criteria is first met until the first date that progressive disease is objectively documented. Responders who do not experience documented progressive disease or death are censored at the time of the last response assessment, and the median and its 95% CI are estimated using Kaplan-Meier method

Progression-Free Survival is analyzed using the Kaplan-Meier method. If a patient does not experience progressive disease or death, then the patient is censored at the time of the last response assessment.

Safety analyses consists of data summaries for clinical and laboratory parameters and AEs. The number and percentage of patients experiencing one or more AEs are summarized by the relationship to study drug and severity based on NCI CTCAE v 5.0. Adverse events are coded using the Medical Dictionary for Regulatory Activities (MedDRA). Laboratory parameters are summarized using descriptive statistics, by post-treatment shifts relative to baseline, and data listings of clinically significant abnormalities. Vital signs and ECG data are summarized using descriptive statistics. Compound 1 plasma concentration data is tabulated with descriptive statistics. Compound 1 exposure parameters are correlated with safety endpoints and antitumor activity.

1. A method for treating an EGFR-mutant cancer in a patient in need thereof comprising administering to the patient a therapeutically effective amount of at least one RET inhibitor and a therapeutically effective amount of at least one EGFR inhibitor.

2. The method of claim 1, wherein the at least one RET inhibitor is chosen from Compound 1 and pharmaceutically acceptable salts thereof.

3. The method of claim 1, wherein the at least one RET inhibitor is chosen from alectinib, apatinib, BOS172738 (DS-5010), cabozantinib (XL184), dovitinib (TKI258), GSK3179106, GSK3352589, lenvatinib, LOXO-292, TPX-0046, SL-1001, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.

4. The method of claim 1, wherein the at least one RET inhibitor is a selective RET inhibitor.

5. The method of any one of claims 1 to 4, wherein the at least one EGFR inhibitor is a selective EGFR inhibitor.

6. The method of any one of claims 1 to 4, wherein the at least one EGFR inhibitor is a third generation EGFR inhibitor.

7. The method of any one of claims 1 to 4, wherein the at least one EGFR inhibitor is chosen from osimertinib and pharmaceutically acceptable salts thereof.

8. The method of any one of claims 1 to 7, wherein the EGFR-mutant cancer is characterized by at least one EGFR mutation chosen from T790M, C797S, and L792H.

9. The method of any one of claims 1 to 8, wherein the EGFR-mutant cancer is further characterized by at least one RET-fusion.

10. The method of claim 9, wherein the EGFR-mutant cancer is further characterized by CCDC6-RET fusion.

11. The method of any one of claims 1 to 10, wherein the EGFR-mutant cancer is lung cancer.

12. The method of claim 11, wherein the lung cancer is chosen from small cell lung cancer and non-small cell lung cancer.

13. The method of any one of claims 1 to 12, wherein the patient is a human.

14. The method of any one of claims 1 to 13, wherein the patient has been previously treated with at least one EGFR inhibitor.

15. The method of any one of claims 1 to 14, wherein the patient has acquired resistance to at least one EGFR inhibitor.

16. The method of any one of claims 1, 2, and 4-15, wherein:
the at least one RET inhibitor is chosen from Compound 1 and pharmaceutically acceptable salts thereof;
the at least one RET inhibitor is orally administered to the patient once daily; and
the therapeutically effective amount of the at least one RET inhibitor is 200 mg to 400 mg of Compound 1 or the weight equivalent of a pharmaceutically acceptable salt thereof.

17. The method of claim 16, wherein the therapeutically effective amount of the at least one RET inhibitor is 200 mg to 300 mg of Compound 1 or the weight equivalent of a pharmaceutically acceptable salt thereof.

18. The method of any one of claims 7 to 17, wherein
the at least one EGFR inhibitor is chosen from osimertinib and pharmaceutically acceptable salts thereof;
the at least one EGFR inhibitor is orally administered to the patient once daily; and
the therapeutically effective amount of the at least one EGFR inhibitor is 80 mg of osimertinib or the weight equivalent of a pharmaceutically acceptable salt thereof.

19. A combination therapy comprising at least one RET inhibitor and at least one EGFR inhibitor.

20. The combination therapy of claim 19, wherein the at least one RET inhibitor is chosen from Compound 1 and pharmaceutically acceptable salts thereof.

21. The combination therapy of claim 19, wherein the at least one RET inhibitor is chosen from alectinib, apatinib, BOS172738 (DS-5010), cabozantinib (XL184), dovitinib (TK1258), GSK3179106, GSK3352589, lenvatinib, LOXO-292, TPX-0046, SL-1001, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.

22. The combination therapy of claim 19, wherein the at least one RET inhibitor is a selective RET inhibitor.

23. The combination therapy of any one of claims 19 to 22, wherein the at least one EGFR inhibitor is a selective EGFR inhibitor.

24. The combination therapy of any one of claims 19 to 22, wherein the at least one EGFR inhibitor is a third generation EGFR inhibitor.

25. The combination therapy of claim 20, wherein Compound 1 is present in an amount of 200 mg to 400 mg.

26. The combination therapy of claim 20, wherein Compound 1 is present in an amount of 200 mg to 300 mg.

27. The combination therapy of any one of claims 19 to 22, 25, or 26, wherein the at least one EGFR inhibitor is chosen from osimertinib and pharmaceutically acceptable salts thereof.

28. The combination therapy of claim 27, wherein osimertinib is present in an amount of 80 mg.

29. A method for treating a patient suffering from an EGFR-mutant cancer, the method comprising:
(a) obtaining a biological sample from the patient;
(b) detecting the presence or absence of at least one RET-fusion in the biological sample; and
(c) administering a combination therapy to the patient if at least one RET-fusion is detected, wherein the combination therapy comprises at least one EGFR inhibitor and at least one RET inhibitor.

30. The method of claim 29, wherein the at least one RET inhibitor is chosen from Compound 1 and pharmaceutically acceptable salts thereof.

31. The method of claim 29, wherein the at least one RET inhibitor is chosen from alectinib, apatinib, BOS172738 (DS-5010), cabozantinib (XL184), dovitinib (TK1258), GSK3179106, GSK3352589, lenvatinib, LOXO-292, TPX-0046, SL-1001, nintedanib, ponatinib, sitravatinib (MGCD516), sorafenib, sunitinib, regorafenib (BAY 73-4506), RXDX-105, vandetanib, XL999, and pharmaceutically acceptable salts of any of the foregoing.

32. The method of claim 29, wherein the at least one RET inhibitor is a selective RET inhibitor.

33. The method of any one of claims 29 to 32, wherein the at least one EGFR inhibitor is chosen from osimertinib and pharmaceutically acceptable salts thereof.

34. The method of any one of claims 29 to 32, wherein the at least one EGFR inhibitor is a selective EGFR inhibitor.

35. The method of any one of claims 29 to 32, wherein the at least one EGFR inhibitor is a third generation EGFR inhibitor.

36. The method of any one of claims 29 to 35, wherein the EGFR-mutant cancer is characterized by at least one EGFR mutation chosen from T790M, C797S, and L792H.

37. The method of any one of claim 29 to 36, wherein the at least one RET-fusion is a CCDC6-RET fusion.

38. The method of any one of claims 29 to 37, wherein the EGFR-mutant cancer is lung cancer.

39. The method of claim 38, wherein the lung cancer is chosen from small cell lung cancer and non-small cell lung cancer.

40. The method of any one of claims 29 to 39, wherein the patient is a human.

41. The method of any one of claims 29 to 40, wherein the patient has been previously treated with at least one EGFR inhibitor.

42. The method of any one of claims 29 to 41, wherein the patient has acquired resistance to at least one EGFR inhibitor.
